(19) United States
(12) Patent Application Publication
(10) Pub. No.: US 2022/0133826 A1
(43) Pub. Date: May 5, 2022

(54) NOVEL COMPONENT FOR CONTROLLING BIOLOGICAL FUNCTION

(71) Applicants: Da Vinci universale Co., Ltd., Tokyo (JP); Asahi Shuzo Co., Ltd., Iwakuni-shi (JP)

(72) Inventor: Asao MURANAKA, Kashiwa-Shi (JP)

(21) Appl. No.: 17/426,788

(22) PCT Filed: Jan. 31, 2020
(86) PCT No.: PCT/JP20/03734

§ 371 Date: Jul. 29, 2021

(30) Foreign Application Priority Data
Feb. 1, 2019 (JP) 2019-017296

(51) Int. Cl. A61K 36/064; A61K 9/127; A61K 8/9728; A61K 8/14; A61Q 19/08; A61P 35/00

(52) U.S. Cl. CPC A61K 36/064; A61K 9/127; A61K 8/9728; A61K 2236/19; A61Q 19/08; A61P 35/00; A61K 2236/53; A61K 8/14

(57) ABSTRACT

An object of the present invention is to prepare a novel food-derived exosome composition and to provide a cancer inhibitor or an agent for preventing decrease in tissue elasticity or an agent for improving elasticity containing a novel active ingredient. As a result of intensive studies on the purification process, the inventors have found that an exosome can be extracted from yeast mash and moromi by a unique production method. The inventors have found that the exosome recovered from the yeast mash and the moromi has a cancer cell proliferation inhibition ability, an ability to promote increase in the amount of granzyme, and an ability to promote expression of collagen and elastin. Accordingly, the inventors provide a composition for treating or preventing cancer and an agent for preventing decrease in tissue elasticity or an agent for improving elasticity, containing exosomes derived from yeast mash and moromi of sake.

### CROSS-REFERENCE TO RELATED APPLICATION

The present international application claims priority based on Japanese Patent Application No. 2019-17296 filed with the Japanese Patent Office on Feb. 1, 2019, and the entire contents of Japanese Patent Application No. 2019-17296 are incorporated herein by reference.

### TECHNICAL FIELD

The present invention relates to medical and cosmetic compositions using a yeast-derived exosome, and the like.

### BACKGROUND ART

Probiotic lactic acid bacteria have been reported to have a preventive effect on metabolic syndrome such as an effect of inhibiting fat accumulation and an effect of improving insulin resistance. As its mechanism, it is considered that exosomes released from orally ingested lactic acid bacteria and absorbed from an intestinal tract interact with tissues, such as adipose tissue and skeletal muscle, locating far from the intestinal tract, and contribute to prevention of metabolic syndrome. Such effects are highly likely to be provided by exosomes released from many bacteria and yeasts which are widely known to be good for human bodies, and also from many vegetables and fruits which are ingested orally, and their usefulness is highly expected.

### SUMMARY OF THE INVENTION

### Problems to be Solved by the Invention

Since yeast mash, row material for producing sake, is obtained by pure mass culture of yeast, the inventors have considered that the yeast mash could be a rich reservoir of exosomes, and for the first time, have focused on an exosome present in a culture solution obtained from the yeast mash that is used as row material in the production process of sake. In addition, for the first time, the inventors have focused on an exosome contained in miso that contains yeast similarly to sake, particularly, miso that has been aged for one year or more. Therefore, the present invention aims to prepare a novel food-derived exosome composition. In another aspect, the present invention is providing compositions for supressing cancer, for preventing loss of tissue elasticity, and for improving elasticity, containing a novel active ingredient.

### Means for Solving the Problems

As a result of intensive studies on the purification process, the inventors have found that an exosome can be extracted by a unique production method. The inventors have examined a change in intracellular granzyme amount in immune cells, and found that the amount of intracellular granzyme increases by addition of exosomes purified from yeast mash and moromi. There was no change in cytokine secretion observed, and no difference in beta-galactosidase staining which is an index of aging in fibroblasts is observed. As another index of aging, expression of collagenase and expression of elastin in aged fibroblasts were evaluated. As a result, it was confirmed that expression levels of collagen and elastin were increased. Accordingly, the inventors have found for the first time that exosomes derived from yeast mash and moromi of sake exert preventing effect on loss of tissue elasticity and improving effect in elasticity.

Furthermore, the inventors have examined changes in gene amounts of collagen type I and elastin, which are known to have an anti-aging effect, in aged fibroblasts, and found that the gene amounts increase depending on the addition of exosomes obtained from miso. In addition, the inventors have found that an immune activity of NK cells is enhanced by adding the exosomes collected from miso.

Thus, the present invention relates to the following:

(1) An exosome extracted from yeast.
(2) The exosome of (1), wherein the yeast is contained in yeast mash or moromi of sake.
(3) An exosome extracted from yeast mash or moromi of sake.
(4) The exosome of claim 2 or 3, wherein the exosome is prepared by obtaining supernatant by centrifuging the yeast mash or moromi of sake at 10,000 to 20,000 kg, and ultracentrifuging the supernatant at 10,000 to 210,000×g, and then obtained precipitate.
(5) The exosome of (1), wherein the yeast is contained in miso.
(6) An exosome extracted from miso.
(7) The exosome of (5) or (6), wherein the miso is aged for one or more years.
(8) The exosome of (7), wherein the miso is aged for three or more years.
(9) A composition comprising the exosome of any one of (1) to (8).
(10) The composition of (9), comprising the exosome of any one of (1) to (8) at 4×108/ml or more, or at 33 ng/ml or more as the protein amount.
(11) The composition according to (9), containing the exosome according to any one of (1) to (8) at 4.7-1011/ml or more.
(12) The composition according to (9), containing the exosome according to any one of (1) to (8) at 7.8×1012/ml or more.
(13) The composition of any one of (9) to (12) containing the exosome as an active ingredient for supressing cancer cell proliferation.
(14) The composition according to (13), wherein the cancer cell is colorectal cancer, prostate cancer, or breast cancer.
(15) The composition of any one of (9) to (12) comprising the exosome as an active ingredient for enhancing granzyme expression from NK cells.
(16) The composition of any one of (9) to (12) comprising the exosome as an active ingredient for treating or preventing cancer.
(17) The composition of any one of (9) to (12) comprising the exosome as an active ingredient for enhancing collagen and/or elastin production.
(18) The composition of any one of (9) to (12) comprising the exosome as an active ingredient for preventing loss of tissue elasticity or for improving tissue elasticity.
(19) The composition for preventing loss of tissue elasticity or for improving tissue elasticity of (18), wherein the tissue is skin.
(20) The composition for preventing loss of tissue elasticity or for improving tissue elasticity of (19), wherein the composition is for cosmetic.
(21) A method for producing the exosome of (1) to (4), comprising:
(a) centrifuging the yeast mash or the moromi of sake at 10,000 to 20,000×g;
(b) ultracentrifuging the supernatant obtained in (a) at 10,000 to 210,000-g; and
(c) collecting the precipitate as an exosome fraction.
(23) A method for producing the exosome according to (5) to (8), comprising:
(a) centrifuging the miso at 10,000 to 20,000×g:
(b) ultracentrifuging the supernatant obtained in (a) at 10,000 to 210,000×g; and
(c) collecting the precipitate as an exosome fraction.

### Advantageous Effects of the Invention

The present invention is based on the success in extracting valuable components for activation of immune system, anti-aging, and body health maintenance. The active ingredient containing an exosome obtained by this unique production method can be applied to products for health maintenance in all forms such as sake and miso as well as other beverages, foods, cosmetics, and pharmaceuticals.

### BRIEF DESCRIPTION OF THE DRAWINGS

FIG. 1 is a photograph showing a result of centrifuging yeast mash and moromi of Dassai at 15,000×g at 4° C. for 15 minutes. The supernatant was used for exosome extraction.

FIG. 2 shows graphs indicating results of measuring the number of particles of the obtained exosome fractions derived from yeast mash (A) and moromi (B) by a nanoparticle analysis system NanoSight (analysis condition: camera level 14, detection threshold 7). A peak of the particle diameter was observed at 95 nm in the exosomes derived from the yeast mash and at 109 nm in the exosomes derived from the moromi.

FIG. 3 shows graphs indicating results of evaluating cell proliferation inhibition and apoptosis after adding each exosome at concentrations of 0.5, 1, and 2 μg/mL to HCT116 cells. For apoptosis, a measurement result of caspase activity is indicated. Further,

FIG. 4 shows graphs indicating results of proliferation inhibition tests for a colorectal cancer cell line HCT116, a lung cancer cell line A549, a breast cancer cell line MDA-MB-231, and a prostate cancer cell line PC-3M. A vertical axis represents a relative value when the value of control (without addition of exosome) is 1.

FIG. 5 shows graphs indicating results of apoptosis inducibility tests for the colorectal cancer cell line HCT116, the lung cancer cell line A549, the breast cancer cell line MDA-MB-231, and the prostate cancer cell line PC-3M). A vertical axis represents a relative value when the value of control (without addition of exosome) is 1.

FIG. 6 shows photographs of results of a migration property test. A linearly white portion at the center is a scratched portion.

FIG. 7 shows a graph indicating results of the migration property test shown in FIG. 6. A vertical axis represents a relative value when the value of control (PBS) is 1.

FIG. 8 shows expression levels of granzyme in NK cells when exosome is added. The upper figure shows photographs of Western blotting showing expression levels of each protein. The lower figure shows a graph that quantifies expression levels by Western blotting.

FIG. 9 shows graphs indicating results of evaluating an action of exosome by NK cells on secretion amounts of three types of cytokines: interferon gamma, interleukin 17 and TNF alpha.

FIG. 10 shows photographs of results of performing beta-galactosidase staining in order to measure an aging retarding ability of exosomes. When cells are stained green, it indicates that the cells are aged.

FIG. 11 is a graph indicating results of evaluating an action of exosomes on expression levels of collagen type I and collagen type III which are important as subcutaneous collagen, and elastin. Bar graphs shown in each group (control (not added), EtOH, 3 μl of moromi, 10 μl of moromi, 30 μl of moromi, 3 μl of yeast mash, 10 μl of yeast mash, and 30 μl of yeast mash, in order from left) indicate results of collagen type I α1 chain, collagen type I α2 chain, collagen type III, and elastin, in order from left. A vertical axis represents a relative value when the value of control is 1.

FIG. 12 shows expression levels of granzyme in NK cells when exosomes are added (n=3). The expression levels by Western blotting were quantified and expressed as average values.

FIG. 13 is a graph indicating results of evaluating an action of exosomes on expression levels of collagen type I and elastin. Bar graphs shown in each group (non-aged, one-year aged, quick brewed, and three-year aged in order from left) indicate results of collagen type I on the left and elastin on the right. A vertical axis represents a relative value when the value of control is 1.

### MODE FOR CARRYING OUT THE INVENTION

1. Preparation of Exosomes Derived from Yeast Mash and Moromi or Miso

Yeast mash is obtained by adding yeast to a mixture of steamed rice, malted rice, and water and culturing, for brewing sake. Fermentation of yeast of the yeast mash proceeds, and a mixture obtained by adding steamed rice, malted rice, and water thereto in several times is moromi. Both yeast mash and moromi are usually used in the industry of sake production, and the production method thereof is also widely known. In particular, yeast mash and moromi used for Dassai are available from Asahi Shuzo Co., Ltd. (2167-4, Shutomachi Osogoe, Iwakuni-shi, Yamaguchi). Extraction of exosomes from yeast mash and moromi can be performed by, for example, (a) centrifuging yeast mash or moromi of sake; (b) ultracentrifuging the supernatant obtained in (a) above; and (c) collecting the precipitate obtained in (b) above as an exosome fraction.

Miso is obtained by mixing malted rice and salt to soybean and fermenting the mixture. “Non-aged miso” refers to miso that has not been subjected to aging processing, and “aged miso” refers to miso that has been produced by natural brewing, aged by allowing to stand using power of natural fermentation of natural yeasts and lactic acid bacteria. The aged miso is called one-year aged miso, three-year aged miso, or the like, depending on the aging period. “Fast-aged miso” refers to miso made in a short period of time by artificially being placed at a certain temperature and subjected to heat treatment, so as to activate action of malted rice (koji) to forcibly ferment miso, and examples thereof include fast-aged miso that has been aged in one month, and the like. The miso in the present specification is preferably aged miso, and examples thereof include miso that has been aged for one to three years, but the miso is preferably aged for one year or more, and more preferably aged for three years or more. Extraction of exosomes from miso can be performed by, for example, (a) centrifuging miso or a suspension of miso (for example, one obtained by dissolving miso in water); (b) ultracentrifuging the supernatant obtained in (a) above; and (c) collecting the precipitate obtained in (b) above as an exosome fraction.

The centrifugation in step (a) can be performed at 10,000 to 20,000×g, 10,000 to 15,000×g, 12,000 to 17,000×g, or 15,000-g. Further, the centrifugation in step (a) can be performed, for example, for 5 minutes or more, 10 minutes or more, or 15 minutes or more. Furthermore, the centrifugation in step (a) can be performed at 4° C. to room temperature. The supernatant obtained in step (a) is used for recovery of exosomes. The supernatant can be subjected to filter filtration (for example, filtration with a 0.65 μm filter) as necessary to further remove residue. The filter filtration can be performed in two stages as necessary.

The ultracentrifugation in step (b) can be performed at 70,000 to 100,000×g, 10,000 to 210,000×g, 50,000 to 200,000×g, or 100,000 to 210,000, g. The ultracentrifugation in step (b) can be performed, for example, for 50 minutes or more, 60 minutes or more, or 70 minutes or more, for example, for 60 minutes to 4 hours. In addition, the ultracentrifugation in step (b) can be performed at 4° C. to room temperature. By collecting the precipitate obtained in step (b) as exosomes, exosomes derived from yeast mash or moromi can be obtained (step C). The recovered exosomes can be washed with PBS or the like as necessary. The washing can be performed by suspending the precipitate in PBS and then centrifuging at 100,000 to 210,000×g to remove the supernatant. The conditions for centrifugation can be set according to the centrifugation in step (b).

Whether or not an exosome is contained in the obtained exosome fraction can be confirmed, for example, by measuring the number of particles using nanoparticle analysis system NanoSight in accordance with the description of examples of the present application, or measuring the amount of contained protein.

Alternatively, it is also possible to acquire a fraction containing an exosome as a main component by using other methods, and an exosome separated and concentrated under such other conditions is also included in the exosome of the present invention. For example, it can also be extracted using a Total Exosome Isolation reagent (Thermo Fisher Scientific) or the like. Ultrafiltration, pelleting.

Therefore, in one aspect, the present invention relates to an exosome extracted from yeast mash or moromi of sake. The exosome of the present invention is preferably an exosome obtained by the above-described method. The exosome is a membrane endoplasmic reticulum with a diameter of 30 to 200 nm surrounded by a lipid bilayer membrane secreted from a cell, and usually contains nucleic acids such as mRNA and miRNA, proteins, and the like. The average particle diameter of the exosome of the present invention is preferably 80 to 200 nm, 100 to 180 nm, 110 to 160 nm, or 118 nm. In addition, the peak diameter of the exosome of the present invention can be preferably 70 to 140 nm, 80 to 130 nm, 90 to 120 nm, or 95 nm.

2. Composition Containing Exosome

In another aspect, the present invention relates to a composition containing the exosome. The content of the exosome in the composition of the present invention is not particularly limited as long as it is a concentration that can achieve the intended use of the composition, and can be set to, for example, 1% by mass or more, 2% by mass or more, 3% by mass or more, 4% by mass or more, 5% by mass or more, 10% by mass or more, 20% by mass or more, 30% by mass or more, 40% by mass or more, or 50% by mass or more. In addition, the amount of the exosome contained in the composition of the present invention can be set to 4×10⁸/ml or more, 5-10⁸/ml or more, 1×10⁹/ml or more, 1×10¹⁰/ml or more, 1×10¹¹/ml or more, or 1×10¹²/ml or more. Alternatively, the amount of the exosome contained in the composition of the present invention can be set to 33 ng/ml or more, 50 ng/ml or more, 100 ng/ml or more, 500 ng/ml or more, 1 μg/ml or more, 5 μg/ml or more, 10 μg/ml or more, 500 μg/ml or more, 1 mg/ml or more, 5 mg/ml or more, 10 mg/ml or more, 50 mg/ml or more, 100 mg/ml or more, 500 mg/ml or more, 1 g/ml or more, or 5 g/ml or more as the amount of protein contained in the exosome.

Components other than the exosome in the composition can be appropriately selected depending on the purpose of use, and can contain, for example, food, a food additive, a cosmetic raw material, a cosmetic additive, a pharmaceutical additive, and the like. Such an exosome composition can be, for example, to produce food (including health food or a nutrient), a cosmetic, or a pharmaceutical, which can be produced by appropriately adding as raw materials for these in the production process. Therefore, the subject of the present invention includes food and cosmetics to which the exosome of the present invention is added (excluding food and cosmetics to which only yeast mash, moromi, and/or miso from which the exosome is derived are added).

The shape of the composition of the present invention is not particularly limited, and can be appropriately selected depending on a use form. Examples thereof include a powder form, a particle form, a granular form, a tablet form, a rod form, a plate form, a liquid form, a candy form, a paste form, a cream form, a capsule form such as a hard capsule or a soft capsule, a caplet form, a tablet-like form, a gel form, a jelly form, a cream form, and the like.

Since the exosome of the present invention has a cancer cell proliferation inhibition effect, the composition containing the exosome can be a composition for inhibiting cancer cell proliferation. In the present specification, the “cancer” includes epithelial malignancies, hematopoietic malignancies derived from spinal cord, and the like, and specifically includes hematopoietic cell malignancies such as lymphoma (Hodgkin's disease, non-Hodgkin lymphoma, and the like) and multiple myeloma; ovarian cancer; breast cancer; mammary gland cancer; uterine cancer such as uterine corpus cancer and cervical cancer; endometrial cancer; ovarian cancer such as non-mucinous ovarian cancer; esophageal cancer; stomach cancer; appendix cancer; colorectal cancer such as colorectal cancer, rectal cancer, and colon cancer; liver cancer such as hepatocellular cancer; gallbladder cancer; bile duct cancer; pancreatic cancer; adrenal cancer; gastrointestinal stromal tumor; mesothelioma such as pleural mesothelioma; head and neck cancer such as laryngeal cancer, throat cancer, and oral cancer (oral floor cancer, gingival cancer, tongue cancer, buccal mucosa cancer, and the like); brain cancer such as ependymoma; salivary gland cancer; sinus cancer (maxillary sinus cancer, frontal sinus cancer, ethmoid sinus cancer, sphenoid sinus cancer, and the like); thyroid cancer; lung cancer; lung adenocarcinoma; osteosarcoma; prostate cancer; testicular tumor or testicular cancer, such as testicular choriocarcinoma; kidney cancer such as renal cell cancer; bladder cancer; sarcomas such as rhabdomyosarcoma; skin cancer; anal cancer; or cancer of the blood such as chronic myelogenous leukemia and acute myelogenous leukemia, or metastatic cancer thereof. Preferably, the cancer is colorectal cancer, prostate cancer, or breast cancer.

In addition, since the exosome of the present invention has an action of promoting granzyme expression by NK cells, the composition containing the exosome can be used for promoting granzyme expression by NK cells. Granzyme is a serine protease that plays a central role in cytotoxic activity by NK cells. Therefore, the composition for promoting granzyme expression by NK cells of the present invention can be used as a pharmaceutical composition for treating or preventing cancer using an ability of NK cells to damage cancer cells, and can also be used as a composition for treating viral or bacterial infection or inflammatory disease.

Furthermore, the exosome of the present invention was confirmed to have a collagen and/or elastin production promoting action. Therefore, the composition containing the exosome can be used for promoting production of collagen and/or elastin. Fiber components such as collagen and elastin are known to play an important role in elasticity of tissues such as skin and blood vessels. Therefore, the composition for promoting production of collagen and/or elastin can be used as a composition for preventing loss of tissue elasticity or improving tissue elasticity. In particular, when the tissue is skin, the composition can also be used for cosmetic. More specifically, it can be used for improving appearance of aged skin, and reducing sagging and/or wrinkles or small wrinkles. The cosmetic composition may be applied to the skin or may be orally ingested into the body.

A use amount or administration amount, and a use method or administration method of the composition of the present invention can be appropriately set depending on the form or purpose of the composition. For example, when ingested as food, an adult can ingest 0.01 mg to 100 g at one time, several times per day.

### EXAMPLES

Hereinafter, the present invention will be specifically described with reference to examples of the present invention, but the present invention is not limited thereto. In addition, all documents cited in this specification are incorporated herein by reference.

For the purpose of evaluating effects on cancer cells, immune cells, and senescent cells, the following experiment was performed on exosomes recovered from yeast mash and moromi used for Dassai of Asahi Shuzo Co., Ltd.

### (Example 1) Preparation of Exosome Sample for Evaluation

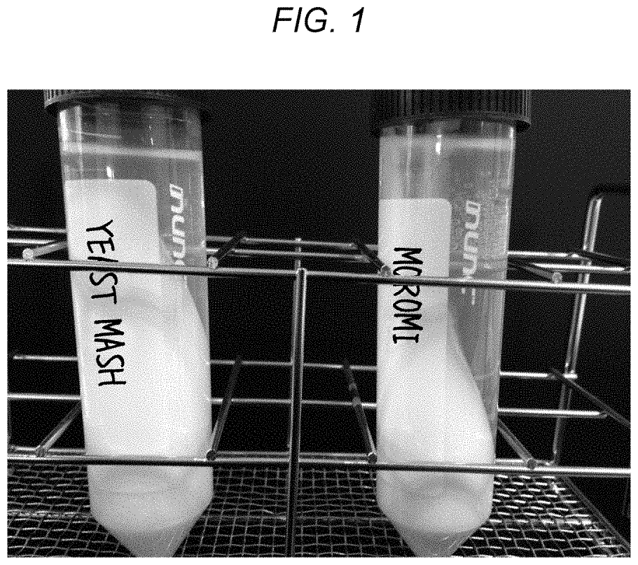

(1) Yeast mash and moromi of Dassai (Asahi Shuzo Co., Ltd.) were each centrifuged at 15,000×g at 4° C. for 15 minutes, and the supernatant was filtered through a 0.65 μm filter (FIG. 1). The supernatant after filtration was ultracentrifuged at 35,000 rpm (100,000×g to 210,000×g) at 4° C. for 70 minutes, using an ultracentrifuge and a SW41 Ti rotor manufactured by Beckman Coulter, Inc. After centrifugation, the supernatant was discarded, and for washing, the precipitate was suspended in PBS, and then ultracentrifugation was performed again under the same conditions. After washing the sample, an exosome fraction remaining on a bottom surface of a tube was recovered in a low adsorption tube. The amount of supernatant after ultracentrifugation was 88 mL for the yeast mash and 176 mL for the moromi. The precipitates of yeast mash and moromi were finally suspended in 170 μL and 340 μL of PBS, respectively.

(2) As non-aged miso, common miso not subjected to aging processing was used. As one-year to three-year aged miso, miso produced by natural brewing, which was prepared by waiting for natural aging in a tub for one to three years using power of natural fermentation of natural yeasts and lactic acid bacteria without artificial heating, was used. As the fast-aged miso, one that was aged in one month by a quick brewing method for making miso in a short period of time was used, the method including artificially being placed at a certain temperature and subjected to heat treatment, so as to activate action of malted rice (koji) to forcibly ferment miso. These misos were centrifuged at 15,000×g at 4° C. for 15 minutes, and the supernatants were each filtered through a 0.65 μm filter (FIG. 1). The supernatant after filtration was ultracentrifuged at 35,000 rpm (100,000×g to 210,000×g) at 4° C. for 70 minutes, using an ultracentrifuge and a SW41 Ti rotor manufactured by Beckman Coulter, Inc. After centrifugation, the supernatant was discarded, and for washing, the precipitate was suspended in PBS, and then ultracentrifugation was performed again under the same conditions. After washing the sample, an exosome fraction remaining on a bottom surface of a tube was recovered in a low adsorption tube and used.

### (Example 2) Measurement of Particle Concentration and Protein Concentration of Prepared Exosome

(1) As for exosome recovered from yeast mash and moromi of Dassai, the yeast mash-derived exosome was diluted 100 times with PBS, and the moromi-derived exosome was diluted 40 times with PBS. The number of particles of each diluted solution was measured with a nanoparticle analysis system NanoSight (analysis condition: camera level 14, detection threshold 7). Regarding the protein concentration, protein quantification was performed using 20 μL of an exosome solution, with Qubit Protein Assay Kit (Invitrogen).

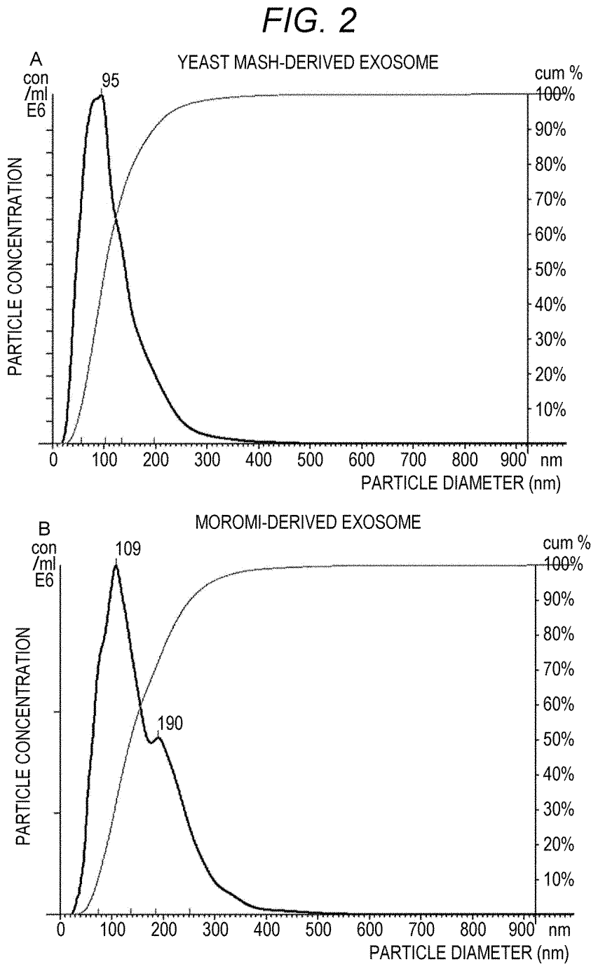

The number of particles and protein concentration of the exosomes recovered from yeast mash and moromi of Dassai using the nanoparticle analysis system are shown in Table 1 below. In addition, FIG. 2 shows a particle size distribution diagram of the particles obtained by the nanoparticle analysis system. The amount of exosomes contained in the yeast mash and the moromi was 3.8×10⁸/ml for the yeast mash and 0.37×10⁸/ml for the moromi. Moreover, the protein concentrations of exosomes contained in the yeast mash and the moromi were 32.1 ng/ml for the yeast mash and 18.1 ng/ml for the moromi.

TABLE 1

| | | | | | Number of |
|---|---|---|---|---|---|
| | Stock | Average | Peak | | particles |
| | solution | particle | particle | Protein | per 1 mL of |
| | concentration | diameter | diameter | concentration | supernatant |
| | (×10⁸) | (nm) | (nm) | (ng/ml) | (×10⁸/mL) |
| Yeast | 1708 | 118 | 95 | 32.1 | 3.3 |
| mash-derived | | | | | |
| exosome | | | | | |
| Moromi- | 190 | 155 | 109 | 18.1 | 0.37 |
| derived | | | | | |
| exosome | | | | | |

(2) After dissolving I g of exosome recovered from each miso in physiological saline, the number of particles was measured by NanoSight (analysis condition: camera level 14, detection threshold 7).

As a result of the measurement, the number of particles was as follows: 1.2×10¹⁰/ml for the non-aged miso, 4.7×10¹¹/ml for the one-year aged miso, 7.8×10¹²/ml for the three-year aged miso, and 2.5×10¹⁰/ml for the fast-aged miso.

### (Example 3) Proliferation Test and Cell Death Test in HCT116 Cell

HCT116 cells (human colon adenocarcinoma-derived cancer cells) were seeded in a 96-well plate at 2,000 cells/well, and the next day, the exosome prepared in Example 1 was added at concentrations of 0.5, 1, and 2 μg/mL. After 3 days, a cell proliferation test was performed to evaluate cell proliferation.

Cell death by apoptosis was evaluated by measuring caspase activity. The medium was removed from the cells of (3) for which the cell proliferation test had ended, and 100 μl of M-PER Mammalian Protein Extraction Reagent (Thermo Fischer Scientific) was added to lyse the cells to obtain a cell lysate, 50 μl of the cell lysate and 50 μl of a solution of Caspase-Glo (registered trademark) 3/7 Reagent (Caspase-Glo (registered trademark) 3/7 Assay Systems, Promega Corporation) were mixed, and the caspase activity was measured by luminescence.

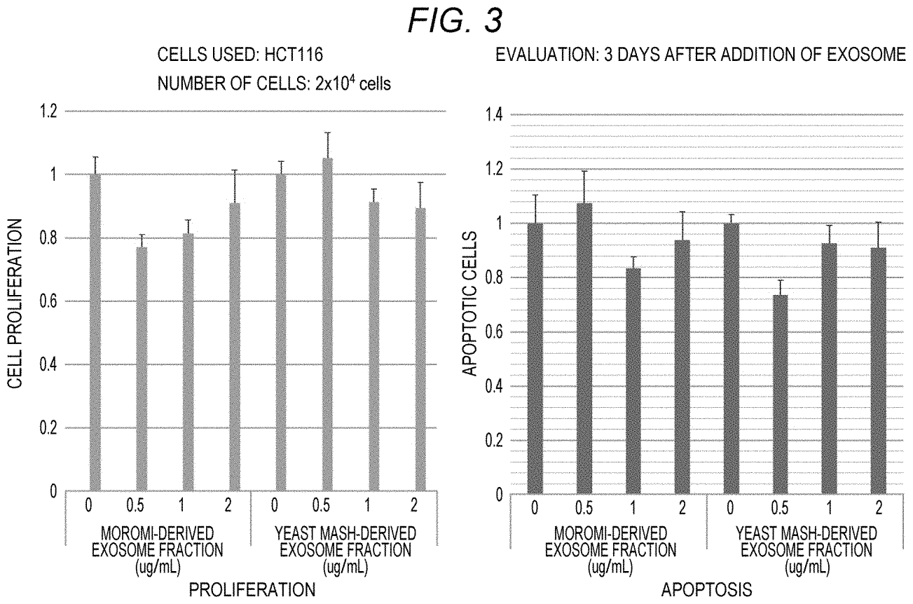

As shown in FIG. 3, when the exosomes derived from the moromi and the yeast mash were added to the colorectal cancer cell line HCT116 at concentrations of 0.5, 1, and 2 μg/mL, proliferation inhibitory activity was observed at 0.5 μg/mL or more for the moromi and at 1 μg/mL or more for the yeast mash, as compared to control (PBS). However, as for apoptosis, no significant difference was observed as compared to the control. Therefore, it was considered that the effect of exosomes derived from the yeast mash and the moromi in the colorectal cancer cell line was cell proliferation inhibitory activity.

### (Example 4) Proliferation Test and Cell Death Test in Various Cancer Cell Types

In order to confirm whether there is a similar effect also in cancer cells other than colon cancer, a proliferation inhibition test and an apoptosis induction ability test were performed for more cancer cell types (colorectal cancer cell line HCT116, lung cancer cell line A549, breast cancer cell line MDA-MB-231, and prostate cancer cell line PC-3M) in the same manner as in Example 3 except that the exosome concentration was set to 2 μg/mL.

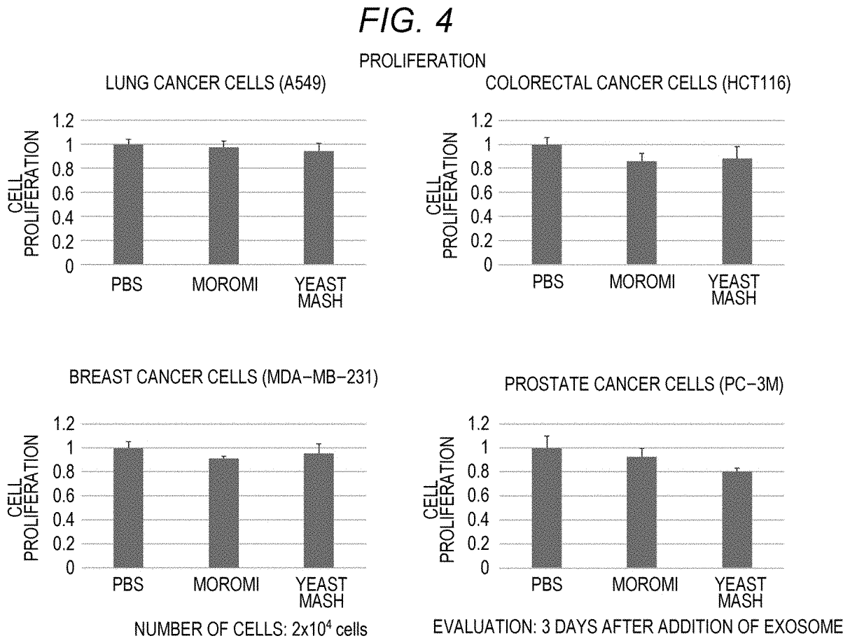

As shown in FIG. 4, a proliferation inhibition effect was reproduced in the colorectal cancer cell line as in Example 2. In addition, an effect equivalent to that of colorectal cancer was also observed in the prostate cancer cell line and the breast cancer cell line, and a proliferation inhibition effect was also observed in lung cancer although being relatively weak.

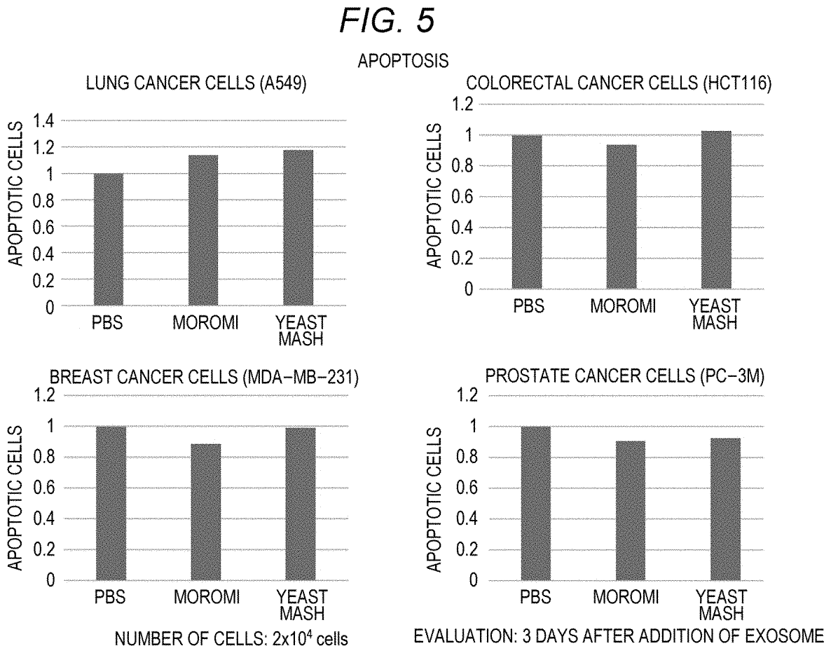

Results of apoptosis measurements are shown in FIG. 5. No significant apoptosis induction was found in all cell lines as a result of evaluation in relative amounts compared to PBS. Therefore, it was reconfirmed that the cell proliferation inhibition effect observed in FIG. 4 was not also due to cell death induction but due to inhibition of cell proliferation.

### (Example 5) Migration Property Test in Cancer Cell

HCT116 cells were seeded in a 24-well plate, and the next day, the inside of the plate was scratched to form a partial space. The cells were photographed on Day 0, Day 1 and Day 2, with the scratched day as Day 0, and how much the scratched part was narrowed by migration of the cells was evaluated.

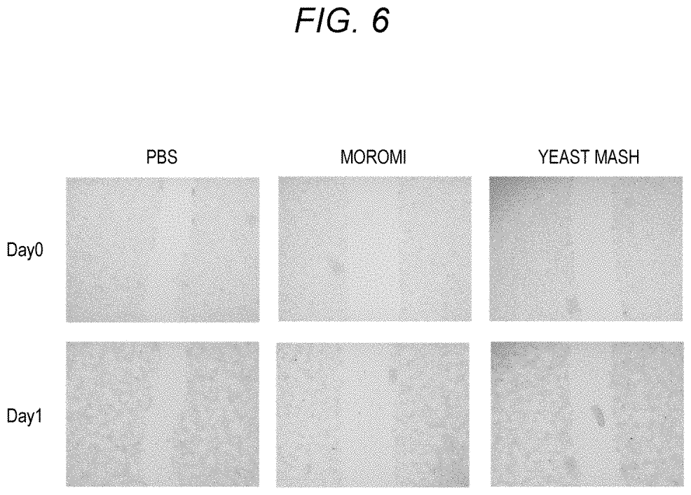

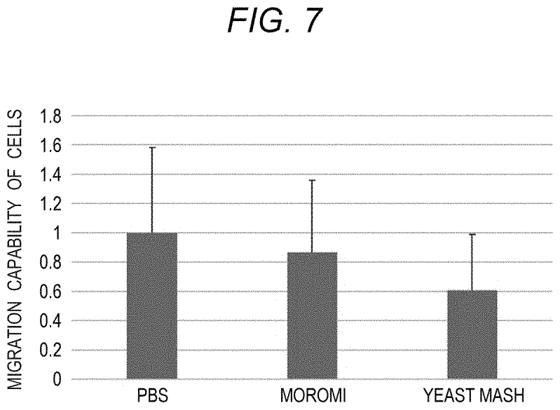

Results of the migration property test are shown in FIG. 6 (photograph) and FIG. 7 (graph). As for the migration ability, a tendency of inhibition with moromi and yeast mash was observed, but a remarkable inhibition effect was not observed.

An exosome fraction was successfully isolated from each of the yeast mash and the moromi. As a result of evaluating proliferation inhibition ability and cell death-inducing ability in cancer cells, a proliferation inhibition effect was observed in the cell lines of prostate cancer, colorectal cancer and breast cancer, and a weak proliferation inhibition effect was observed in the lung cancer cell line. Since it was confirmed that cell death of these cell lines was not induced, it was found that the exosomes derived from the yeast mash and the moromi have an effect of inhibiting only proliferation of cells without causing cancer cells to die. In addition, with respect to the migration ability of cancer cells, a tendency of inhibition was observed, but no remarkable change was observed. Based on the above, an effect of inhibiting proliferation of cancer cells was observed in the exosomes derived from the yeast mash and the moromi, but it was considered that this effect was not so strong as to induce cell death as an anticancer agent, but was an effect of gently stopping the proliferation of cancer cells.

TABLE 2

Human cancer cells

| | Colorectal | Breast | Prostate | Lung |
|---|---|---|---|---|
| Effect of Dex | cancer | cancer | cancer | cancer |
| Cell proliferation | +++ | + | +++ | +/− |
| inhibition | | | | |
| Cytocidal effect | − | − | − | − |
| Infiltration | − | − | − | − |
| inhibition ability | | | | |

### (Example 6) Expression Analysis of Granzyme in Immune Cell

(1) NK-92MI cells, which are NK cells that can be cultured for a long time, were seeded in a 24-well plate at 25.0000 cells/well, and the exosomes prepared in Example 1 were added so as to be 3, 10, or 30μ/L (for the miso exosome, approximately 100 particles per cell, i.e. 2×10⁷ particles per well). After 2 days, the cells were recovered, protein extraction was performed, and the expression level of the protein was measured by Western blotting.

(2) Results of Yeast Mash Exosome

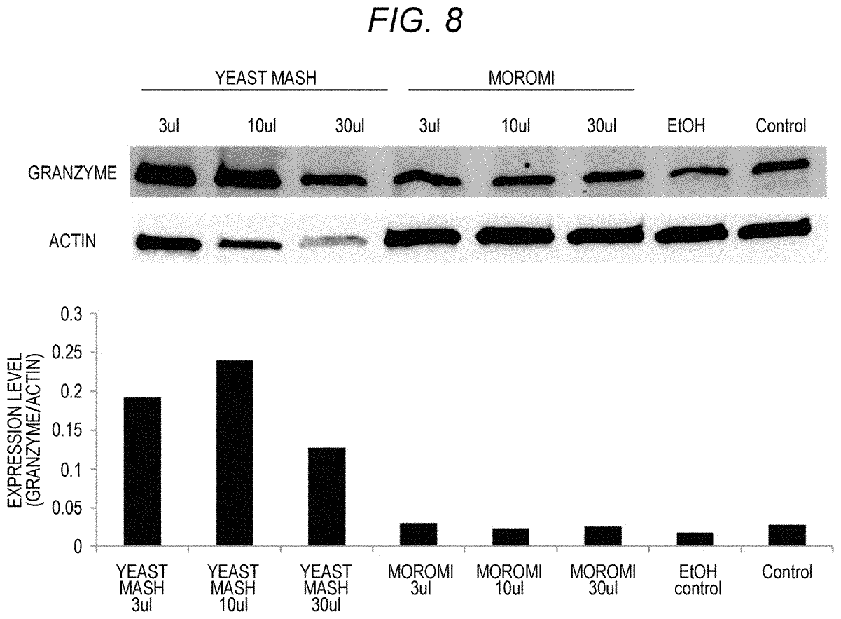

As shown in FIG. 8, an increase in the expression level of granzyme in NK cells was observed by adding the yeast mash exosome. The upper figure of FIG. 8 shows photographs of the Western blotting for evaluating the expression levels of protein, and the lower figure shows a graph that quantifies the expression levels. Based on the above, it is considered that the yeast mash exosome contributes to a cytocidal effect by increasing expression of granzyme in NK cells. Regarding the moromi exosome, no significant change was observed in the expression level of granzyme.

(3) Results of Miso Exosome

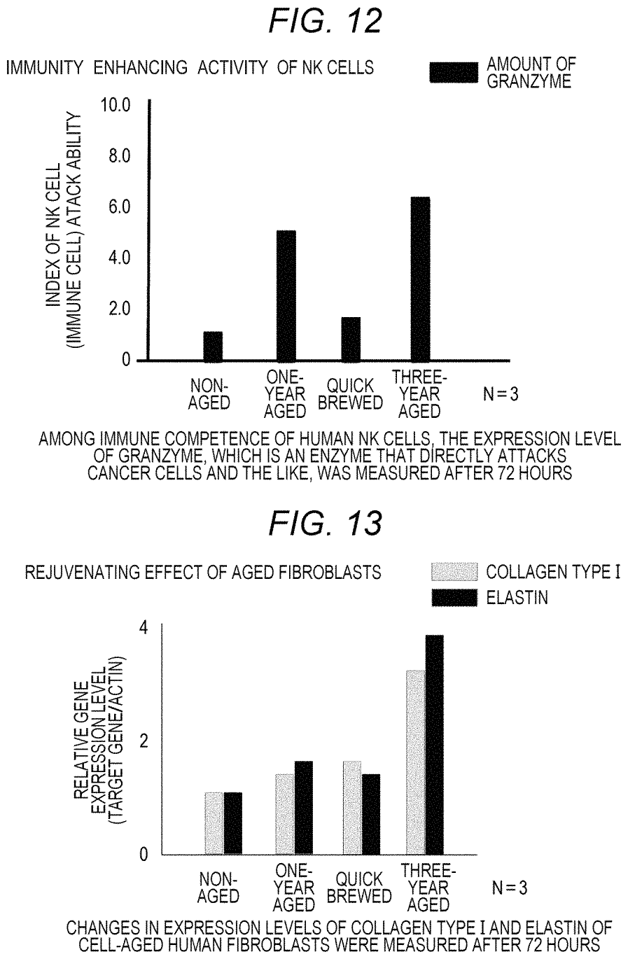

As shown in FIG. 12, an increase in the expression level of granzyme in NK cells was observed by adding the exosomes of one-year and three-year aged miso. Therefore, it is considered that the exosome of miso aged for one year or more contributes to the cytocidal effect by increasing the expression of granzyme in NK cells.

### (Example 7) Measurement of Secretion of Cytokine in Immune Cell

As cytokine secretion of NK cells, secretion of three types of main cytokines: interferon gamma, interleukin 17, and TNF alpha, were evaluated. NK-92MI cells were seeded in a 24-well plate at 25,0000 cells/plate, and the exosomes were added so as to be 3, 10, or 30μ/L. After 2 days, the culture supernatant was recovered, and centrifuged at 300 g for 3 minutes to recover the supernatant. The measurement of interferon gamma, interleukin 17, and TNF alpha in the supernatant was performed using an ELISA kit (R&D systems).

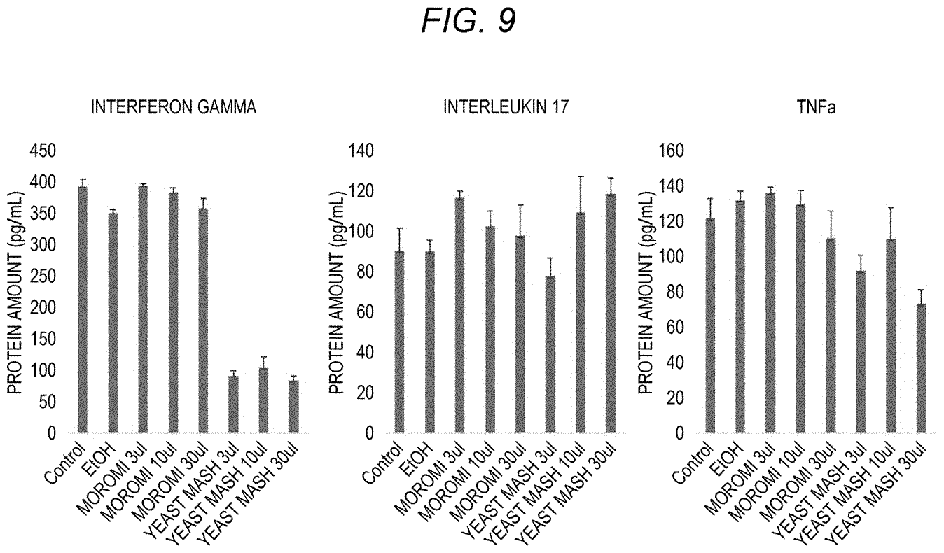

Results are shown in FIG. 9. Although a large increase in the secretion was not observed in any of the cytokines, the secretion of interferon gamma decreased in the yeast mash exosome.

### (Example 8) Beta-Galactosidase Staining in Senescent Cell

Beta-galactosidase staining was performed as a method for evaluating senescent cells. SPiDER-βGal (DOJINDO LABORATORIES), which is known to be highly sensitive, was used for beta-galactosidase staining. Specifically, fibroblasts (P8) to which exosomes were added so as to be 3, 10, or 30μ/L were seeded in a 60 mm dish at 2×10⁵ cells/dish and passaged twice to age the cells. The exosomes were added so as to be 3, 10, or 30μ/L each time the cells were passaged. Thereafter, aged fibroblasts (P10) were seeded in a 35 mm glass base dish. On the next day, the medium was removed by suction, and the dish was washed once with 2 ml of HBSS. After 72 hours from the start of the culture, 2 ml of a 4% paraformaldehyde/PBS solution was added to immobilize the cells at room temperature for 3 minutes. 2 ml of the 4% paraformaldehyde/PBS solution was removed by suction, and washing was performed twice with 2 ml of HBSS. 2 ml of SPiDER-βGal working solution (DOJINDO LABORATORIES) was added, and the dish was allowed to stand at 37° C. for 30 minutes. Thereafter, washing was performed twice with 2 ml of HBSS, and observation was performed with a confocal laser microscope.

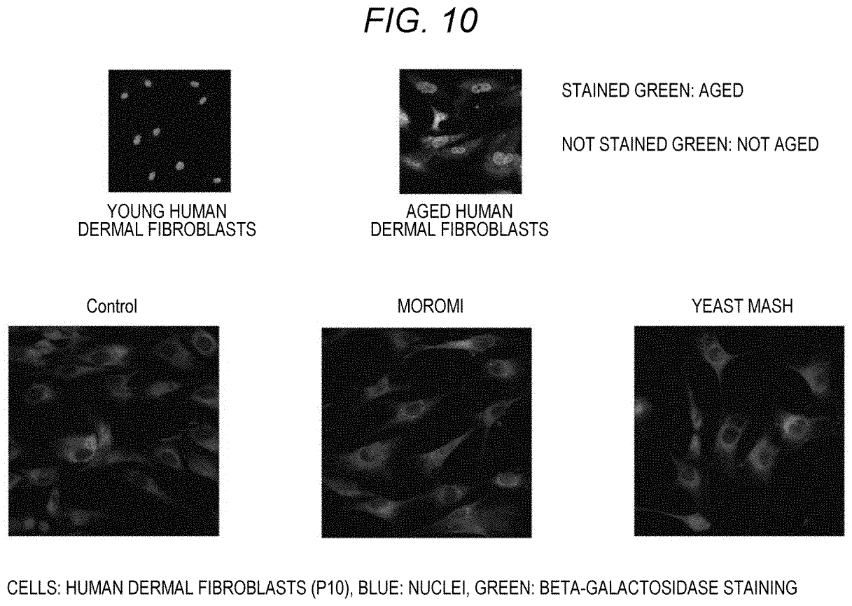

Results are shown in FIG. 10. Since the cells were stained green in both the control and the exosome-added cells, it was shown that the exosomes derived from the moromi and the yeast mash could not stop aging of the cells.

### (Example 9) Measurement of Expression Levels of Collagenase and Elastin in Senescent Cell

(1) Changes in the expression levels of collagen and elastin with reduced activity in aged fibroblasts due to exosome addition were evaluated. Aged fibroblasts (P10) were seeded in a 24-well plate at 100,000 cells/well, and the next day, the exosomes were added so as to be 3, 10, or 30μ/L (for the miso exosome, approximately 100 particles per cell, i.e. 2×10⁷ particles per well). After 2 days, RNA extraction was performed using Qiagen miRNeasy mini kit. Using the extracted RNA as a sample. RT-PCR was performed using High-Capacity cDNA Reverse Transcription Kit (Applied Biosystems). Taqman Universal Master Mix II (of Applied Biosystems) was used for quantitative PCR. As primers/probes for detection of collagenase and elastin, evaluation was performed using Taqman method of Applied Biosystems. Primers/probes of collagen type I alpha 1 chain, collagen type I alpha 2 chain, collagen type III, and elastin were used.

(2) Results of Yeast Mash and Moromi Exosomes

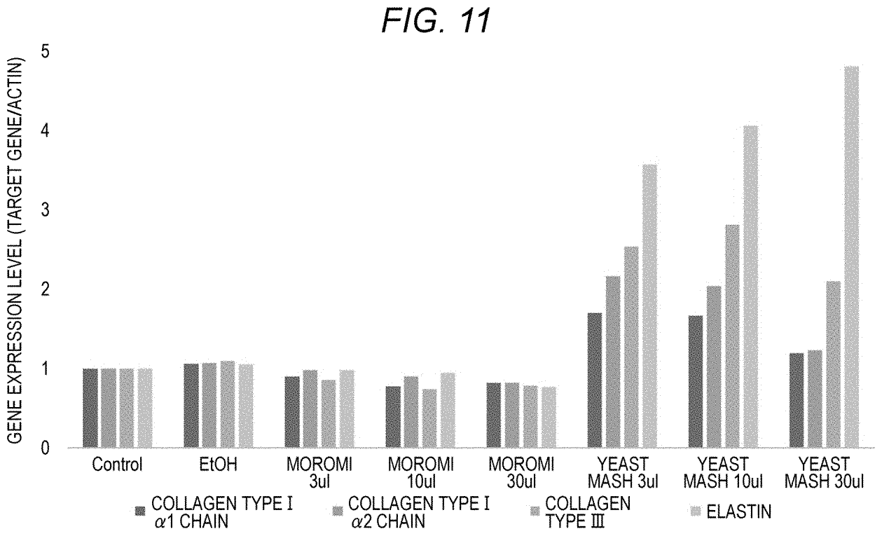

The expression levels of collagen I and collagen III, which are important as subcutaneous collagen, and elastin, were evaluated. Results are shown in FIG. 11. It was found that the expression levels of collagen type I, collagen type III, and elastin were all increased by adding the yeast mash exosome. On the other hand, when the moromi exosome was added, no change was observed in the expression levels of these substances.

(3) Results of Miso Exosome

The expression levels of collagen type 1 and elastin were evaluated. Results are shown in FIG. 13. It was found that the expression levels of collagen type I and elastin were all increased by adding the miso exosome. In particular, when the three-year aged exosome was added, the expression levels of these substances remarkably increased.

As described above, the exosome fractions were isolated from the yeast mash and the moromi as well as the miso, and the functions in immune cells and senescent cells were evaluated. Regarding immune cells, attention was paid to NK cells having a cytocidal effect on cancer cells. As a result, it was found that the amount of granzyme, which is known to have a cytocidal effect, in the cell increased by the addition of the yeast mash exosome. This result suggests that the NK cells more effectively kill many cancer cells when attacking cancer cells. On the other hand, no significant change was observed in the secretion of cytokines that attract other immune cells. Although the secretion of interferon gamma in the yeast mash exosome is reduced, it can be considered that it is a stage in which the NK cells enhance a cytocidal activity, also from the fact that the secretion is inversely correlated with the expression level of granzyme

On the other hand, with respect to the evaluation of aging in the fibroblasts, it was not possible to stop the aging of fibroblasts in both of the yeast mash exosome and the moromi exosome. On the other hand, since production of collagen and production of elastin in aged fibroblasts were enhanced by the addition of the yeast mash exosome, it was suggested that the yeast mash exosome has an action of enhancing the production of collagen and elastin which are reduced by aging of the skin.

Based on the above, it was found that the exosomes derived from yeast (yeast mash, moromi, and miso) enhance the cytocidal effect of NK cells by increasing the expression level of granzyme. In addition, it is considered that for senescent cells, the exosomes can contribute to prevention of decrease in elasticity and improvement in elasticity in the skin by increasing the expression levels of collagen and elastin.

TABLE 3

| | Moromi | Yeast mash |
|---|---|---|
| Anti-cancer action | + | + |
| Immunity enhancement effect | − | ++ |
| Anti-aging action | − | − |
| Skin-beautifying effect | − | +++ |

1-22. (canceled)

23. An exosome extracted from yeast.

24. The exosome of claim 23, wherein the yeast is contained in yeast mash or moromi of sake.

25. An exosome extracted from yeast mash or moromi of sake.

26. The exosome of claim 24, wherein the exosome is prepared by obtaining supernatant by centrifuging the yeast mash or moromi of sake at 10,000 to 20,000×g, and ultracentrifuging the supernatant at 10,000 to 210,000×g, and then obtained precipitate.

27. The exosome of claim 23, wherein the yeast is yeast contained in miso.

28. An exosome extracted from miso.

29. The exosome of claim 27, wherein the miso is aged for one or more years.

30. The exosome of claim 29, wherein the miso is aged for three or more years.

31. A composition comprising the exosome of claim 23.

32. The composition of claim 31, comprising the exosome extracted from yeast at 4×10⁸/ml or more, or at 33 ng/ml or more as a protein amount.

33. The composition of claim 31, comprising the exosome extracted from yeast at 4.7×10¹¹/ml or more.

34. The composition of claim 31, comprising the exosome extracted from yeast at 7.8×10¹²/ml or more.

35. A method for suppressing cancer cell proliferation, comprising administering the composition of claim 31 which contains the exosome as an active ingredient.

36. The method of claim 35, wherein the cancer cell is colorectal cancer, prostate cancer, or breast cancer.

37. A method for enhancing granzyme expression from NK cells, comprising administering the composition of claim 31 which contains the exosome as an active ingredient.

38. A method for treating or preventing cancer, comprising administering the composition of claim 31 which contains the exosome as an active ingredient.

39. A method for enhancing collagen and/or elastin production, comprising administering the composition of claim 31 which contains the exosome as an active ingredient.

40. A method for preventing loss of tissue elasticity or for improving tissue elasticity, comprising administering the composition of claim 31 which contains the exosome as an active ingredient.

41. The method of claim 40, wherein the tissue is skin.

42. The method of claim 41, wherein the composition is for cosmetic.

43. A method for producing the exosome of claim 24, comprising:
(a) centrifuging the yeast mash or the moromi of sake at 10,000 to 20,000×g;
(b) ultracentrifuging the supernatant obtained in (a) at 10,000 to 210,000×g; and
(c) collecting a precipitate as an exosome fraction.

44. A method for producing the exosome of claim 27, comprising:
(a) centrifuging the miso at 10,000 to 20,000×g;
(b) ultracentrifuging the supernatant obtained in (a) at 10,000 to 210,000×g; and
(c) collecting a precipitate as an exosome fraction.
